(19) United States
(12) Patent Application Publication
(10) Pub. No.: US 2012/0315683 A1
(43) Pub. Date: Dec. 13, 2012

(54) ETHANOL PRODUCTION FROM LIGNOCELLULOSIC BIOMASS WITH RECOVERY OF COMBUSTIBLE FUEL MATERIALS

(76) Inventors: Nathan Mosier, West Lafayette, IN (US); Michael R. Ladisch, West Lafayette, IN (US); Jerry B. Warner, Alexandria, VA (US)

(21) Appl. No.: 13/446,559

(22) Filed: Apr. 13, 2012

Related U.S. Application Data
(63) Continuation of application No. PCT/US10/52503, filed on Oct. 13, 2010.
(60) Provisional application No. 61/251,059, filed on Oct. 13, 2009.

(51) Int. Cl. C12P 7/10

(52) U.S. Cl. 435/165

(57) ABSTRACT

Described are processes for producing a product, such as ethanol, from lignocellulosic biomass, and producing a burnable fuel material from byproducts. The burnable fuel material can be burned on site to produce energy to feed back into the production process.

### REFERENCE TO RELATED APPLICATION

This application is a continuation of International Application No. PCT/2010/052503, filed Oct. 13, 2010, which claims the benefit of U.S. Provisional Application No. 61/251,059, filed Oct. 13, 2009, now abandoned, each of which is hereby incorporated by reference in its entirety.

### BACKGROUND

The present invention relates generally to the utilization of lignocellulosic biomass as a source for the production of ethanol, and in certain embodiments, the recovery of energy bearing materials that are not consumed in the ethanol formation for use as burnable materials.

As further background, increasing emphasis has been placed in recent years upon finding ways to efficiently produce fuels from renewable, non-petroleum resources. In one field of interest, fuel ethanol has been produced by fermentation of biomass feedstocks derived from plants. Currently, fuel ethanol is commercially produced from feedstocks of cornstarch, sugar cane and sugar beets. These materials, however, find significant competing uses in the food industry, and their expanded use to make fuel ethanol is met with increased prices and disruption of other industries. Alternative fermentation feedstocks and viable technologies for their utilization are thus highly sought after.

Lignocellulosic biomass materials are available in large quantities and are relatively inexpensive. Such materials are available in the form of agricultural wastes such as corn stover, corn fiber, wheat straw, barley straw, oat straw, oat hulls, canola straw, soybean stover, grasses such as switch grass, miscanthus, cord grass, and reed canary grass, forestry wastes such as aspen wood and sawdust, and sugar processing residues such as bagasse and beet pulp. Cellulose from these materials is converted to sugars, which are then fermented to produce the ethanol.

A problem with using lignocellulosic biomass materials for ethanol production is disposing of the residual lignins and other non-ethanol forming materials. These materials would traditionally be sent to waste water treatment facilities for disposal. In this age of recycling and awareness of the environment, allowing these organic materials, which may contain potential energy value to be disposed in this manner is not the most environmentally friendly or efficient use. In certain of its aspects, the present invention is addressed to this problem of utilization of the residues from biomass ethanol formation.

### SUMMARY

In one embodiment, provided is a process for converting biomass into ethanol. The process includes processing a first portion of lignocellulosic biomass to produce ethanol and non-ethanol forming materials, the processing including heating; collecting at least a portion of the non-ethanol forming materials left after the processing; drying the collected non-ethanol forming materials; and burning the non-ethanol forming materials to generate heat. The heat can then be supplied to a processing of a second portion of lignocellulosic biomass to produce ethanol.

In another embodiment, provided is a process for recovery of a burnable fuel material from the conversion of lignocellulosic biomass to ethanol. The process includes processing lignocellulosic biomass to produce a product stream comprising ethanol, solids and other organic material, removing the ethanol from the product stream leaving a non-product stream, isolating the solids from the non-product stream, removing water from the non-product stream producing a concentrated non-product stream, and drying the solids and concentrated non-product stream to produce a burnable fuel.

In another embodiment, provided is a process for recovery of burnable fuel material. The process includes obtaining lignocellulosic biomass from plant material grown within about 50 miles of a lignocellulosic biomass processing facility, processing a first amount of the biomass to produce ethanol, collecting by-product materials from the processing of the biomass, processing the by-product materials to form a burnable fuel material, and using the burnable fuel material on-site at the lignocellulosic biomass processing facility to produce energy. Additional embodiments of the invention as well as features and advantages thereof will be apparent from the descriptions herein.

### BRIEF DESCRIPTION OF THE FIGURES

FIG. 1 is a schematic representation of steps used in the process of converting lignocellulosic biomass to ethanol including steps for the recovery of non-ethanol material for use as burnable fuel.

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications in the illustrated device, and such further applications of the principles of the invention as described herein being contemplated as would normally occur to one skilled in the art to which the invention relates.

As used herein, the term “lignocellulosic biomass”, is meant to refer to any type of biomass comprising lignin and cellulose such as, but not limited to, non-woody plant biomass, agricultural wastes and forestry residues and sugar-processing residues. For example, the cellulosic feedstock can include, but is not limited to, grasses, such as switch grass, cord grass, rye grass, miscanthus, mixed prairie grasses, or a combination thereof; sugar-processing residues such as, but not limited to, sugar cane bagasse and sugar beet pulp; agricultural wastes such as, but not limited to, soybean stover, corn fiber from grain processing, corn stover, oat straw, rice straw, rice hulls, barley straw, corn cobs, wheat straw, canola straw, oat hulls, and corn fiber; and forestry wastes, such as, but not limited to, recycled wood pulp fiber, sawdust, hardwood, softwood, or any combination thereof. Further, the lignocellulosic biomass may comprise lignocellulosic waste or forestry waste materials such as, but not limited to, paper sludge, newsprint, cardboard and the like. Lignocellulosic biomass may comprise one species of fiber or, alternatively, a lignocellulosic biomass feedstock may comprise a mixture of fibers that originate from different lignocellulosic materials.

Typically, the lignocellulosic material will comprise cellulose in an amount greater than about 2%, 5% or 10% and preferably greater than about 20% (w/w) to produce a significant amount of glucose. The lignocellulosic material can be of higher cellulose content, for example at least about 30% (w/w), 35% (w/w), 40% (w/w) or more. Therefore, the lignocellulosic material may comprise from about 2% to about 90% (w/w), or from about 20% to about 80% (w/w) cellulose, or from 25% to about 70% (w/w) cellulose, or about 35% to about 70% (w/w) cellulose, or more, or any amount in between.

Prior to pretreatment, the lignocellulosic biomass can be mechanically processed to increase its surface area. Such mechanical processing may include, for example, reducing the biomass to a particulate by grinding, milling, agitation, shredding, or other types of mechanical action.

The lignocellulosic biomass can be used to create a pumpable slurry in combination with a suitable liquid, preferably an aqueous medium. The aqueous medium may be water alone, but in other embodiments can include additives to enhance the pretreatment process such as acids or bases to adjust or maintain the pH of the aqueous medium. The aqueous slurry of the lignocellulosic biomass will typically be relatively highly concentrated in solids. In certain embodiments, the aqueous slurry will be comprised at least about 10 grams per liter (g/l) of lignocellulosic biomass solids, preferably at least about 50 g/l, more preferably at least about 100 g/l, and typically in the range of about 100 g/l to about 500 g/l. It will be understood, however, that other solids concentrations may be used in broader aspects of the invention.

Aspects of the present invention are also applicable to systems which employ dilute acid pretreatment processes. Suitable acids for these or other purposes herein include for example inorganic or organic acids, e.g. sulfuric, hydrochloric, phosphoric nitric, acetic, citric or formic acid. Suitable bases for these purposes include for example alkali or alkaline earth metal hydroxides, e.g. sodium or potassium hydroxide, or other hydroxide bases such as ammonium hydroxide. In certain preferred forms, the aqueous medium will be adjusted initially and/or during a thermal pretreatment process by the addition of acid or base to provide a pH that is near neutral, so as to avoid the occurrence of any significant acid- or base-catalyzed autohydrolysis of the lignocellulosic material, for example a pH in the range of about 5 to about 8. Additional information as to suitable conditions for pH-controlled lignocellulosic biomass pretreatments is found in U.S. Pat. No. 5,846,787, which is hereby incorporated herein by reference in its entirety. Other additives that may be present in the aqueous biomass slurry include, as illustrations, surfactants, e.g. vegetable oils such as soybean oil, canola oil, and others, to serve as intercalating agents.

After pretreatment, the biomass is subjected to a cellulase enzyme to produce a fermentable material. In this regard, a cellulase enzyme is an enzyme that catalyzes the hydrolysis of cellulose to products such as glucose, cellobiose, and/or other cellooligosaccharides. Cellulase enzymes may be provided as a multienzyme mixture comprising exo-cellobiohydrolases (CBH), endoglucanases (EG) and beta-glucosidases (betaG) that can be produced by a number of plants and microorganisms. The process of the present invention can be carried out with any type of cellulase enzymes, regardless of their source; however, microbial cellulases provide preferred embodiments. Cellulase enzymes can, for example, be obtained from fungi of the genera Aspergillus, Humicola, and Trichoderma, and from the bacteria of the genera Bacillus and Thermobifida.

Following this hydrolysis of the pretreated biomass, an aqueous medium containing the resulting sugars can be subjected to fermentation to produce ethanol. The fermentation of the sugars to produce ethanol can be conducted with any of a wide variety of fermentive microorganisms such as yeast or bacteria, including genetically modified versions thereof, and using known techniques. The ethanol can then be purified from the fermented medium, for example by distillation. The material left after ethanol removal if not utilized in some way would be sent to a waste water treatment facility. However, this material has significant energy content, and could provide a more efficient use if it were recovered and used for its energy content instead of sending for waste treatment where it would have a significant disposal cost and provide no benefit.

In certain modes of practice, during the processing of biomass, including lignocellulosic biomass, unhydrolyzed solids and other non-ethanol forming materials, typically including lignin, organic components derived from biomass, phenolic compounds, yeast, enzymes, and lignin degradation products can be separated from liquids as a byproduct, for example by filtration, centrifugation or in a settling tank producing wet solids. Solids may be recovered at various stages of the biomass to ethanol process, such as after the hydrolysis stage but before fermentation, after fermentation but before distillation or after distillation. Wet solids from any of these isolation steps could be combined and dried or dried separately to form burnable solids. These burnable solids may be used locally or on-site to produce energy, such as for use in a boiler or generator that supplies heat to be used in the processing of subsequently-processed lignocellulosic biomass, e.g. in heating aqueous mixtures of lignocellulosic biomass for pretreatment as described herein and/or in heating mixtures undergoing fermentation to ethanol as described herein.

Soluble non-ethanol forming materials can be recovered from liquid streams after ethanol has been removed by distillation by concentrating the liquid streams containing these materials through unit operations such as a multiple effect evaporation unit, a hybrid multiple effect evaporation unit, a flash distillation column or some other unit operation that can be used to remove a more volatile material from the residue. The wet solids previously isolated and the concentrated liquid can be dried to produce solids possessing significant energy content. The wet solids and concentrated liquids can be dried separately or after they have been combined. The solids from any of the drying processes can be further processed to produce a material that can be used as a burnable fuel. In some cases the concentrate will result in evaporated solids would form a binder to hold together particles of the material. The processing can result in particles or pellets of burnable materials that can be easily handled and readily transported. The processing of the material from the drying process may include pelletizing, grinding, sieving, extruding or any other method that may be used to produce an easily handled and transported material. This material can be used in a variety of instances that require a burnable material such as a coal furnace. In one aspect, the burnable materials could be used locally or on-site to generate energy or to provide a source of fuel for such things as boilers or generators, on another type of equipment that uses solid burnable fuel. In another aspect, the energy generated on-site by burning the material recovered from the processing of lignocellulosic biomass to ethanol may be utilized to supply heat to the subsequent processing of additional lignocellulosic biomass to ethanol, such as in the pretreatment step, hydrolysis step or fermentation step described herein. In still other embodiments, the dried solids burnable fuel material can be shipped to another location to be burned to produce energy, for example at a site relatively local to the ethanol-producing facility in which the dried solids were produced (e.g. within about 100 miles).

In certain modes of practicing the present invention, improved energy sufficiency and efficiency for ethanol production and/or a lower carbon footprint are provided in operating a cellulose to ethanol plant. The solids themselves derived from plant matter in fact will yield energy and CO₂ where the CO₂ will be integrated back into plant matter by virtue of the Calvin cycle. Consequently, the minimal environmental footprint of such a plant, where burnable solids are recovered from the process and are then used for a coal fire plant either on-site or off-site is clear. In a certain modes of practice, the biomass material is grown and harvested within about 100 miles, more preferably within about 40 or 50 miles of the facility where the biomass is processed to provide ethanol. When the biomass is obtained locally, and the solids that are generated are used locally (within a 40 to 50 mile radius), this is an example of localized boiler fuel derived from products from a cellulose to ethanol process. The CO₂ impact on the global environment would be minimal with the all the CO₂ that is generated coming from a local source of renewable biomass. In this vein, in certain embodiments, the processing facility can be operated completely or predominantly (e.g. at least about 80%) upon such locally-grown biomass material in order to minimize or reduce its CO₂ impact.

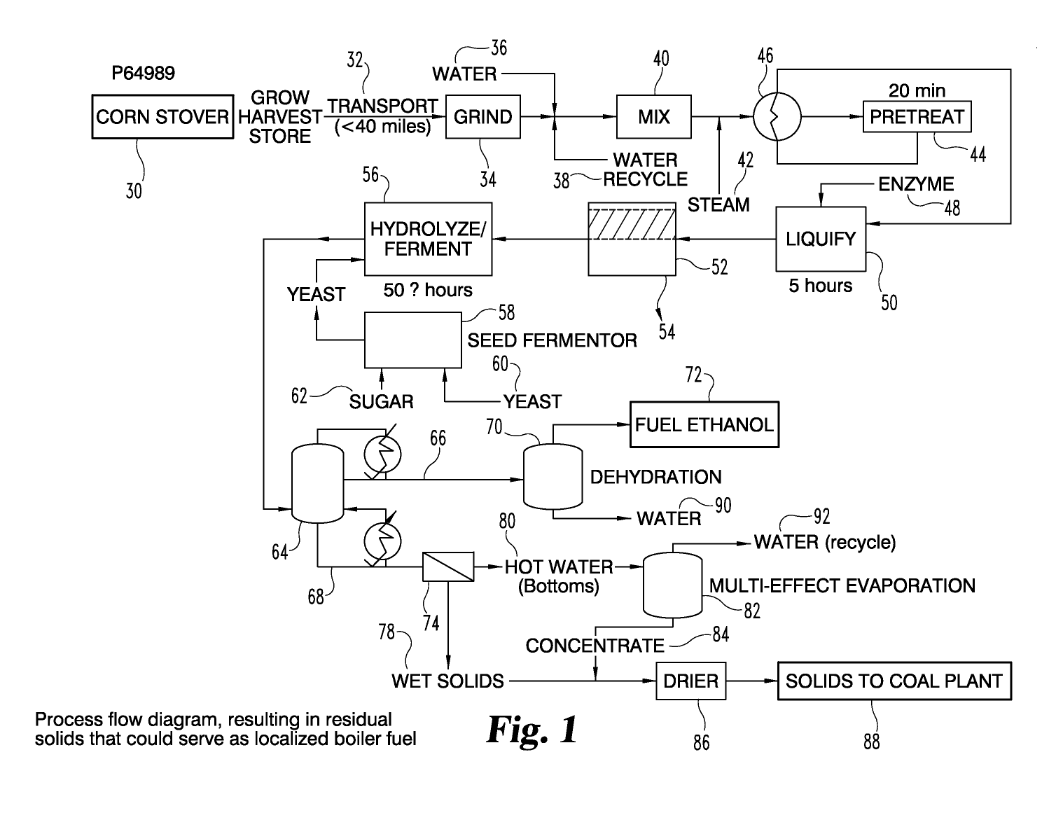

With reference now to FIG. 1, one embodiment of processing lignocellulose biomass including isolating non-ethanol forming solids for use as a burnable fuel is shown. Corn stover 30 or other lignocellulosic biomass is grown, harvested and stored within about five hundred miles, or within one hundred miles, or preferably within about forty miles of the processing facility. The lignocellulosic biomass is transported 32 to the facility where it is ground 34, water 36 which may include recycled water 38 from the process is added to the ground lignocellulosic biomass and mixed 40 to give a mixture of consistency appropriate for the pretreatment system. The mixture of ground lignocellulosic biomass and water is heated to appropriate temperatures for pretreatment and held at that temperature for an appropriate time. The pretreatment heating of the mixture can, for example, be conducted in a series of two or more heat exchangers, e.g. as described in copending application Nos. 61/076,019 filed Jul. 26, 2008 and 61/076,034 filed Jul. 26, 2008, both of which are hereby incorporated by reference in their entirety. Further descriptions of the pretreatment of lignocellulosic biomass are found in U.S. Pat. No. 5,846,787, which is hereby incorporated herein by reference in its entirety. Pretreatment temperatures can include 120° C. to 220° C., or 150° C. to 200° C., or 160° C. to 190° C. and pretreatment times at which the mixture is held at these temperatures range from 10 minutes to 60 minutes, or 10 minutes to 40 minutes, or minutes to 20 minutes. After pretreatment is complete, the mixture is cooled, and a cellulase enzyme 48 is added and the material in the mixture is allowed to liquefy 50 by holding the mixture with the cellulase enzyme present for up to 5 hours. The liquefied material may optionally be further processed to separate out 52 (e.g. by press or centrifuge) a liquid stream 54 containing dissolved soluble material. Liquid Stream 54 can be routed to multi-effect evaporator 82 (discussed below) to result in the recovery of dissolved solids from stream 54 in a concentrated stream 84, (also discussed below), which is ultimately added to wet solids 78 and provides additional fuel value to a dried solids product 88. Such recovery of dissolved solids from stream 54 can also reduce the level of organics in waste streams resulting from the overall process.

The remaining mixture is sent to a hydrolysis and fermentation reactor 56. Yeast is added to the reactor 56 from a yeast source 58 where yeast may be grown by the addition of supplementary yeast 60 and sugar 62. The mixture is allowed to hydrolyze and ferment for about 50 hours. During the hydrolysis and fermentation it is desirable that a significant fraction of the cellulosic material is fermented to ethanol. This may be achieved through a combination of enzyme addition, yeast addition and pH adjustment to enable the enzyme to hydrolyze the cellulose to sugars, and the yeast to ferment the sugars to ethanol. Preferred pH is between 5 and 6.5.

After hydrolysis and fermentation has occurred, a distillation 64 is carried out and the overhead product 66 containing ethanol and some water is sent to a dehydration system 70. The dehydration system produces an ethanol product stream 72, and a water stream 90 which may be recycled to earlier parts of the process, thus saving water. A solid slurry and liquid stream 68 containing non-ethanol forming material from the distillation 64 is sent through a solid-liquid separation device 74 that removes the solids 78 from the liquid 80. Any solid-liquid separation device normally used in industry could be utilized to carry out this separation such as filters, pressure filters, vacuum filters, settling tanks or centrifuges. The liquid 80 from the solid-liquid separator 74 is then subject to a multiple effect evaporator 82 where a water stream 92 is removed and a concentrated stream 84 is produced. The water stream 92 removed may be recycled to earlier parts of the process, thus saving water. Other methods could be used in place of a multiple effect evaporator 82 to remove water and other volatiles from the process liquid, for example a flash column, distillation column, or low pressure evaporator. The concentrated stream 84 of non-ethanol forming material left after water has been removed is mixed with the wet solids 78 from the solid-liquid separator 74 and are then fed to a dryer 86. The solids 88 from the dryer 86 are processed to be used as burnable fuel for such facilities as in a coal fire plant.

To provide a further understanding of aspects of the present invention, and their advantages, the following specific Example is given. It will be understood that this Example is illustrative, and not limiting, of the invention.

### Example 1

A total weight of 1600 lbs of corn stover is ground and mixed with water to give a total water content of 10,670 lbs (including moisture from the corn stover). The amount of water added to the corn stover is adjusted in order to give the total ratio which represents a concentration of 13% weight/weight, or 15% weight/volume of lignocellulosic material in water. This mixture is pretreated by heating it to a temperature between 160° C. and 190° C., and for hold times ranging from 10 minutes to 20 minutes. After pretreatment the material is cooled, a cellulase enzyme is added, and the material is liquefied by holding for 5 hours. The liquefied material is further processed to press out dissolved material in a water stream, yielding 8,936 lbs of liquid with a concentration of 60 grams/liter soluble material. The remaining material at a level of 461 grams/liter (weight/volume) basis is then fed to a hydrolysis/fermentation reactor where yeast is added to ferment the sugars that are generated. The yeast itself is obtained from a seed fermenter. Fermentation time is approximately 50 hours until a significant fraction of the cellulosic material is converted to ethanol. This fermentation is achieved through a combination of enzyme addition and yeast addition where the pH is adjusted to enable the enzyme to hydrolyze the cellulose to sugars, and the yeast to ferment the sugars to ethanol. Preferred pH is between 5 and 6.5.

After hydrolysis and fermentation has occurred, distillation is carried out and the overhead product containing some water is then metered to a dehydration system where the ethanol product is collected and a water stream is obtained which may be recycled to earlier parts of the process, thus conserving water. The solid slurry and liquid stream left from the distillation is filtered where solids are removed from the liquid. The liquid stream from the filter is then concentrated by multiple effect evaporation. The concentrated stream from the multiple effect evaporator is then combined with the wet solids collected by the filter, and these are then dried and processed into particles or pellets for easy handling. The form of these processed material are selected to be a form suitable for use as a burnable solid fuel such as for a coal fire plant.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

1. A process for converting biomass into ethanol, comprising:
processing a first portion of lignocellulosic biomass to produce ethanol and non-ethanol forming materials, the processing including heating;
collecting at least a portion of the non-ethanol forming materials left after the processing; drying the collected non-ethanol forming materials;
burning the non-ethanol forming materials to generate heat; and
supplying the heat to a processing of a second portion of lignocellulosic biomass to produce ethanol.

2. The process of claim 1 where the lignocellulosic biomass is from a source less than 100 miles from a location of said processing steps.

3. The process of claim 1 where the lignocellulosic biomass is from a source less than 40 miles from a location of said processing steps.

4. The process of claim 1 where said burning is conducted in a boiler.

5. A process for the isolation of energy containing material from process streams of the process of converting biomass into ethanol comprising:
isolating solid residues from a process stream produced from the production of ethanol from lignocellulosic biomass;
concentrating non-ethanol forming materials in a liquid process stream left after ethanol and solids removal producing a concentrate;
and drying the solid residues and concentrate to produce a burnable fuel.

6. The process of claim 5 where the solid residues and the concentrate are combined before drying.

7. The process of claim 5 where the lignocellulosic biomass is from a source less than 100 miles from a location of said production of ethanol.

8. The process of claim 5 where the lignocellulosic biomass is from a source less than 40 miles from a location of said production of ethanol.

9. The process of claim 5 where the concentrating comprises multiple effect evaporation.

10. The process of claim 5 where the concentrating comprises flash distillation.

11. The process of claim 5 further comprising burning the burnable material on-site at a location of said production of ethanol to produce energy.

12. The process of claim 5 further comprising processing the burnable material to a particulate form.

13. The process of claim 12 where the particulate form is a pellet form.

14. A process for recovery of a burnable fuel material from the conversion of lignocellulosic biomass to ethanol comprising:
processing lignocellulosic biomass to produce a product stream comprising ethanol, solids and other organic material;
removing the ethanol from the product stream leaving a non-product stream;
isolating the solids from the non-product stream;
removing water from the non-product stream producing a concentrated non-product stream;
and drying the solids and concentrated non-product stream to produce a burnable fuel.

15. The process of claim 14 where the lignocellulosic biomass is from a source less than 100 miles from a location of said processing.

16. The process of claim 14 where the lignocellulosic is from a source less than 40 miles from a location of said processing.

17. The process of claim 14 further comprising processing the burnable fuel to a particulate form.

18. The process of claim 17 where the particulate form is a pellet form.

19. The process of claim 14 further comprising burning the burnable fuel on-site at a location of said processing lignocellulosic biomass, to produce energy.

20. A process for recovery of burnable fuel material comprising:
obtaining lignocellulosic biomass from plant material grown within about 50 miles of a lignocellulosic biomass processing facility;
processing a first amount of the biomass to produce ethanol;
collecting by-product materials from the processing of the biomass;
processing the by-product materials to form a burnable fuel material; and
burning the burnable fuel material on-site at the lignocellulosic biomass processing facility to produce energy.

21. The process of claim 20, wherein the energy is heat, and wherein the heat is supplied to a processing of a second amount of the biomass to produce ethanol.
